(19) United States
(12) Patent Application Publication
(10) Pub. No.: US 2025/0235670 A1
(43) Pub. Date: Jul. 24, 2025

(54) Securement Systems for Vascular Devices

(71) Applicant: Solventum Intellectual Properties Company, Maplewood, MN (US)

(72) Inventor: James M. Sieracki, Plymouth, MN (US)

(21) Appl. No.: 18/700,917

(22) PCT Filed: Oct. 10, 2022
(86) PCT No.: PCT/IB2022/059703

§ 371 Date: Apr. 12, 2024

Related U.S. Application Data
(60) Provisional application No. 63/270,632, filed on Oct. 22, 2021.

(51) Int. Cl. A61M 25/02

(52) U.S. Cl. CPC A61M 25/02; A61M 2025/0246; A61M 2025/0273; A61M 2025/028; A61M 2205/0205; A61M 2205/0216

(57) ABSTRACT

Attachment systems for attaching a vascular device to a patient includes an adhesive and a polymeric hardgood article for holding a vascular device. One surface of the polymeric hardgood article (140,240,340) is attached to the adhesive layer (110A,130A,220,230). The opposite surface of the polymeric hardgood article has a hole that passes through the entire article and a pathway adjacent to the hole. The pathway has a channel (343,443) extending to an edge surface, or a series of posts (541), where the series of posts define the pathway.

### SUMMARY

Disclosed herein are attachment systems for attaching vascular devices to a patient, kits that contain attachment systems, and methods for attaching vascular devices to a patient.

In some embodiments, the attachment system for attaching a vascular device to a patient comprises a base adhesive attachment layer with a first major surface and a second major surface, where the first major surface comprises an adhesive surface suitable for attachment to mammalian skin, and a polymeric hardgood article for holding a vascular device. The polymeric hardgood article has a first major surface and a second major surface, the first major surface is in contact with and attached to the second major surface of the base adhesive attachment layer. The second major surface of the polymeric hardgood article comprises a hole that passes through the first major surface, and a pathway adjacent to the hole, where the pathway comprises a channel extending to an edge surface of the second major surface, or a series of posts, where the series of posts define the pathway.

Also disclosed are kits that comprise the attachment system described above and a cover film article.

Methods for using an attachment system for a vascular device comprises providing a vascular device. providing an attachment system for a vascular device, where the attachment system has been described above, inserting a vascular device into a patient, where the vascular device passes through the hole in the hardgood article, attaching the attachment system to the patient, and threading the vascular device through the pathway adjacent to the hole.

### BRIEF DESCRIPTION OF THE DRAWINGS

The present application may be more completely understood in consideration of the following detailed description of various embodiments of the disclosure in connection with the accompanying drawings.

FIG. 1A is a cross sectional view of an embodiment of a vascular securement article of this disclosure.

FIG. 1B is a cross sectional view of another embodiment of a vascular securement article of this disclosure.

FIG. 2 is a cross sectional view of an embodiment of a vascular securement article of this disclosure disposed on mammalian skin.

FIG. 3 is a top view of an embodiment of a hardgood element of a vascular securement article of this disclosure.

FIG. 4 is a tope view of an embodiment of a vascular securement article of this disclosure.

FIG. 5 is a tope view of an embodiment of another vascular securement article of this disclosure.

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Various medical devices are attached to a patient. For example, tubing, monitors, sensors, or catheters are secured to skin. To limit irritation, dislodgement, and potential exposure to infection, the medical devices should be securely attached to the patient. Adhesives and adhesive tapes are commonly used to secure devices to skin. Very strong adhesives can cause trauma to skin upon removal. A very gentle adhesive will remove from the skin easily but might not have sufficient strength to secure the medical device. Therefore, there is a need for medical adhesive articles that adhere strongly and yet are removable without causing skin damage or pain.

Among the recent developments are the use of stretch removability to permit removal of the adhesive article from the skin. In these articles, a film backing is used that has an extensible substrate with a securing adhesive that can secure to a surface and a tab to stretch the extensible substrate to remove the adhesive from the underlying surface. Stretching the extensible substrate will release the securing adhesive from the underlying surface easily and without trauma. Therefore, a strong adhesive can be used.

This mechanism is known as “stretch release”. Stretch release tapes are known for use in securing items to surface, such as walls. For example, 3M COMMAND tape is a stretch release tape with a foam substrate coated on both surfaces with a strong adhesive. In U.S. Patent Ser. No. 62/910,667 filed on Oct. 4, 2019 such a stretch releasable medical tape article is described.

Among the medical articles that are attached to patients are vascular devices. Vascular devices are devices that are inserted into veins for diagnostic or therapeutic reasons, such as blood sampling, central venous pressure readings, administration of medication, fluids, total parenteral nutrition (TPN) and blood transfusions. Among vascular devices are central venous access devices (CVADs) or central venous catheters (CVCs), devices that are inserted into the body through a vein to enable the administration of fluids, blood products, medication and other therapies to the bloodstream. CVADs can be inserted into the subclavian or jugular vein (implanted ports, tunneled catheters), or can be inserted into one of the peripheral veins of the upper extremities, called peripherally inserted central catheters (PICCs).

Catheters of various types (e.g. venous, arterial) are routinely placed in patients in hospitals and are typically secured using sutures, tape, a dressing or a securement device. Movement of an inserted catheter can lead to irritation of the vein and other complications, requiring premature removal of the catheter and insertion of the catheter in a different vein. This results in a waste of resources in both medical supplies and labor, as well as extended patient care and discomfort. Further, since each patient has a limited number of catheter insertion sites, it is undesirable to repeatedly replace a catheter at a new insertion site on a patient this process expends the available insertion sites.

Because there are many different manufacturers and hub sizes vary even within the same manufacturer, securement devices that can accommodate a wide variety of catheters are preferred by clinicians. Typically, these products often utilize aggressive adhesives that must be removed using an adhesive-remover or alcohol. Therefore, the need remains for an attachment system for a wide range of vascular devices that is easy to use, provides stable securement, and is gentle to remove without adverse reactions to skin.

Disclosed herein is a system including a hardgood design that allows for adequate securement of many different catheters and a stretch-removable film base that enables skin-friendly removal of the system from the patient. The system is designed to allow for easy and intuitive application, provides visual access to the insertion site, and offers hassle-free removal when desired by the clinician. Also disclosed herein is a kit for securing a vascular device. The kit comprises an attachable holding device and a cover film. The attachment securement device comprises a polymeric hard good article for holding a vascular device and a base adhesive attachment layer. Additionally, methods of using the attachment system for a vascular device are also disclosed.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates. The gel adhesives may have some properties in common with pressure sensitive adhesives, but they are not pressure sensitive adhesives. “Hydrogel adhesives” are gel adhesives that have water as the fluid contained within the crosslinked matrix.

The term “(meth)acrylate” refers to monomeric acrylic or methacrylic esters of alcohols. Acrylate and methacrylate monomers or oligomers are referred to collectively herein as “(meth)acrylates”. Materials referred to as “(meth)acrylate functional” are materials that contain one or more (meth)acrylate groups.

The terms “siloxane” and “siloxane-based” as used herein refer to polymers or units of polymers that contain siloxane units. The terms silicone or siloxane are used interchangeably and refer to units with dialkyl or diaryl siloxane (—SiR₂O—) repeating units.

The term “adjacent” as used herein when referring to a pathway and a hole, at least a portion of the pathway is in contact with the hole. When the term adjacent refers to two layers, it means that the two layers are in proximity with one another with no intervening open space between them. They may be in direct contact with one another (e.g. laminated together) or there may be intervening layers.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

The term “alkyl” refers to a monovalent group that is a radical of an alkane, which is a saturated hydrocarbon. The alkyl can be linear, branched, cyclic, or combinations thereof and typically has 1 to 20 carbon atoms. In some embodiments, the alkyl group contains 1 to 18, 1 to 12, 1 to 10, 1 to 8, 1 to 6, or 1 to 4 carbon atoms. Examples of alkyl groups include, but are not limited to, methyl, ethyl, n-propyl, isopropyl, n-butyl, isobutyl, tert-butyl, n-pentyl, n-hexyl, cyclohexyl, n-heptyl, n-octyl, and ethylhexyl.

The term “aryl” refers to a monovalent group that is aromatic and carbocyclic. The aryl can have one to five rings that are connected to or fused to the aromatic ring. The other ring structures can be aromatic, non-aromatic, or combinations thereof. Examples of aryl groups include, but are not limited to, phenyl, biphenyl, terphenyl, anthryl, naphthyl, acenaphthyl, anthraquinonyl, phenanthryl, anthracenyl, pyrenyl, perylenyl, and fluorenyl.

Unless otherwise indicated, the terms “optically transparent”, and “visible light transmissive” are used interchangeably, and refer to an article, film or adhesive that has a high light transmittance over at least a portion of the visible light spectrum (about 400 to about 700 nm). Typically, optically transparent articles have a visible light transmittance of at least 90% and a haze of less than 10%.

Unless otherwise indicated, “optically clear” refers to an adhesive or article that has a high light transmittance over at least a portion of the visible light spectrum (about 400 to about 700 nm), and that exhibits low haze, typically less than about 5%, or even less than about 2%. In some embodiments, optically clear articles exhibit a haze of less than 1% at a thickness of 50 micrometers or even 0.5% at a thickness of 50 micrometers. Typically, optically clear articles have a visible light transmittance of at least 95%, often higher such as 97%, 98% or even 99% or higher.

Disclosed herein are attachment systems for attaching vascular devices to a patient. In some embodiments, the attachment system comprises a base adhesive attachment layer with a first major surface and a second major surface, where the first major surface comprises an adhesive surface suitable for attachment to mammalian skin, and a polymeric hard good article for holding a vascular device. The polymeric hard good article has a first major surface and a second major surface, the first major surface is in contact with and attached to the second major surface of the base adhesive attachment layer. The second major surface comprises a hole that passes through the first major surface, and a pathway adjacent to the hole. The pathway comprises a channel extending to an edge surface of the second major surface or comprises a series of posts, where the series of posts define the pathway.

The base adhesive attachment layers comprise a multi-layer stretch removable article. In some embodiments, the multi-layer stretch removable article comprises a multi-layer extensible backing substrate, and two adhesive layers. In some embodiments, the multi-layer extensible backing substrate comprises at least three layers. These layers comprise a first plastic skin layer with a first major surface and a second major surface, a core elastomeric layer with a first major surface and a second major surface, and a second plastic skin layer with a first major surface and a second major surface.

The core elastomeric layer is in contact with the second major surface of the first plastic skin layer and the first major surface of the second plastic skin layer. Examples of suitable multi-layer extensible backing substrates are described in U.S. Patent Ser. No. 62/910,667. In the multi-layer extensible backing substrate, the core comprises an elastomeric material. Elastomeric means that the material exhibits at least some ability to return at least in part to its original shape or size after forces causing the deformation are removed. Examples of elastomeric material include elastomeric polymer, SEBS, SEPS, SIS, SBS, polyurethane, ethyl vinylacetate (EVA), ethyl methyl acrylate (EMA), ultra low linear density polyethylene (ULLDPE), hydrogenated polypropylene, and combinations or blends thereof.

The skin layers of the multi-layer extensible backing substrate may be the same or different, typically they are the same. In some embodiments, the skin layers comprise a plastically deforming material. Plastic deformation means that the material undergoes a permanent change in shape or size when subjected to a stress exceeding a particular value (the yield value). Examples of plastically deforming materials include polypropylene, polyethylene, high density polyethylene (HDPE), low density polyethylene (LDPE), linear low density polyethylene (LLDPE), polyurethane, ethyl vinylacetate (EVA), ethyl methyl acrylate (EMA), and combinations or blends thereof.

The core layer and skin layers can be bonded to one another using any suitable mechanism including, for example, coextruding the core and the skin layer(s), co-molding, extrusion coating, joining through an adhesive composition, joining under pressure, joining under heat, and combinations thereof.

The extensible substrate has a low Fn Modulus. The Fn Modulus is the force required to elongate the extensible substrate. The Fn Modulus is the force (F) required to elongate the test specimen a specified percent (n). Fn Modulus is usually recorded for elongation of 10%, 50%, and 100% for comparison of films.

Particularly suitable extensible substrates for application on skin has a F (50%) Modulus less than 20 N per 25.4 mm width. In some embodiments, the extensible substrate has a F (50%) Modulus less than 10 N per 25.4 mm width at 50% strain, or even a F (50%) Modulus less than 5 N per 25.4 mm width.

Elongation is the maximum percent of strain reached by a test sample to the point of breakage. The extensible substrate has a low elongation to minimize the distance needed to stretch and pull the extensible substrate. A very high elongation means the extensible substrate will have a high distance it stretches before releasing from the surface. Excessive stretching is an inconvenience in settings where space is constrained. Modulus and elongation typically are measured under ambient conditions (25° C. and 50% relative humidity) by a method based upon PSTC-31, ASTM D882 and D3759 Test Methods. The test can be performed on a constant rate of extension/tensile tester (such as an Instron, Zwick or equivalent). Typically, the extensible substrate 110 has an elongation of less than 600%.

In some embodiments, the extensible substrate has an elongation of less than 500%, an elongation less than 300%, or even an elongation of at least 100%.

It is desirable that a majority of the deformation of the extensible substrate is plastic deformation and that its elastic deformation is minimized. Elasticity is the ability of a deformed material body to return to its original shape and size when the forces causing the deformation are removed. In contrast, plasticity is the property of a solid body whereby it undergoes a permanent change in shape or size when subjected to a stress exceeding a particular value (the yield value). A highly elastic film may present a hazard during stretching to remove the film from a surface because as the film stretches, a significant amount of energy is stored in it. If the stretching film is not secured, upon the release of the film from the surface this energy will accelerate the film, and any attached device, toward the hand stretching the film, possibly resulting in injury or a broken device.

Plastic deformation is calculated using the original length of the extensible substrate (Lo), the length the extensible substrate reached under applied stress (Ls) and the length the extensible substrate relaxed to after the stress was removed (Lr).

% Plastic deformation=(Lr−Lo)/(Ls−Lo)×100

For example, a one-inch (Lo) length of film stretched to five inches (Ls), which relaxes to a length of four inches (Lr) when the applied stress is removed, has a plastic deformation of 75%. ((4−1)/5−1)×100).

Plastic deformation of the extensible substrate reduces the energy released upon the removal of stress, since the energy input to extensible substrate is consumed by the re-ordering the film morphology rather than stored as potential energy in elastic structures. Less stored energy improves safety during the removal of devices held in place with an extensible substrate. The combination of low modulus and large degree of plastic deformation makes one-handed stretch and removal of the extensible substrate from the surface possible, without the hazard of the extensible substrate from being snapped painfully into the removing hand.

The extensible substrate has a plastic deformation of at least 50%. In some embodiments, the extensible substrate has a plastic deformation of at least 60%, or even at least 70%.

The multi-layer extensible backing substrate may further comprise a tab at a perimeter portion of the extensible substrate. This tab is non-tacky and is designed to be grasped by a user to affect the stretch removal of the adhesive article. In some embodiments, the tab is a portion of and edge of the film backing that is free of adhesive. In other embodiments, the tab is a cover over the securing adhesive to create a portion of the first major surface that is free of a tacky adhesive. Therefore, a user can easily pull the extensible substrate from the underlying surface to remove the extensible substrate from the surface to which it is adhered.

In some embodiments, the stretch removable article further comprises a second tab on the opposite end of the article from the first tab. The second tab may be the same or different from the first tab.

The base adhesive attachment layer also comprises two adhesive layers, disposed on the multi-layer extensible backing substrate. These adhesive layers are described as a first adhesive layer and second adhesive layer. The first adhesive layer is in contact with the first plastic skin layer, and the first adhesive layer is suitable for attachment to mammalian skin. The second adhesive layer is in contact with the third plastic skin layer, and the second adhesive layer attaches the multi-layer stretch removable article to the polymeric hard good article.

The first adhesive layer may be continuous or discontinuous and is suitable for adhering the stretch removable article to human skin. Typically, the securing adhesive is a pressure sensitive adhesive. In some embodiments, the adhesive may be a gel adhesive. Siloxane-based gel adhesives are particularly suitable gel adhesives. Among the suitable classes of pressure sensitive adhesives include rubber-based adhesives, siloxane-based adhesives, and (meth)acrylate-based adhesives. In some embodiments, the adhesive can include tackified rubber adhesives, such as natural rubber; olefins; siloxanes, such as silicone polyureas; synthetic rubber adhesives such as polyisoprene, polybutadiene, and styrene-isoprene-styrene, styrene-ethylenebutylene-styrene and styrene-butadiene-styrene block copolymers, and other synthetic elastomers; and tackified or untackified (meth)acrylate adhesives such as copolymers of isooctylacrylate and acrylic acid, which can be polymerized by radiation, solution, suspension, or emulsion techniques; polyurethanes; silicone block copolymers; and combinations of the above. The adhesive can be, for example, any of the adhesives described in PCT Patent Publication Nos. 2015/035556, 2015/035960, and US 2015/034104. In some embodiments, the adhesive includes additives such as tackifiers. Some exemplary tackifiers include polyterpene, terpene phenol, rosin esters, and/or rosin acids.

The first adhesive layer may comprise a variety of additives as long as the additives do not interfere with the adhesive properties of the adhesive layer. Particularly suitable additives include anti-microbial additives, rendering the first adhesive layer into an anti-microbial adhesive layer.

A wide range of anti-microbials are suitable for use in the first adhesive layer. Suitable anti-microbial additives include benzalkonium chloride, copper, silver, quaternary ammonium compounds, polyhexamethylene biguanide, chlorhexidine gluconate, fatty acid monoesters.

The second adhesive layer may be continuous or discontinuous and attaches the multi-layer stretch removable article to the polymeric hard good article. Typically, this layer is a pressure sensitive adhesive. The second adhesive layer is often different from the first adhesive layer, since the second adhesive layer is not a mammalian skin adhesive. A wide range of pressure sensitive adhesives are suitable. Pressure sensitive adhesives useful for the second adhesive layer include tackified natural rubbers, synthetic rubbers, tackified styrene block copolymers, polyvinyl ethers, acrylics, poly-alpha-olefins, and silicones.

The first adhesive layer may comprise a variety of additives as long as the additives do not interfere with the adhesive properties of the adhesive layer. Particularly suitable additives include anti-microbial additives, rendering the first adhesive layer into an anti-microbial adhesive layer.

A wide range of anti-microbials are suitable for use in the first adhesive layer. Suitable anti-microbial additives include benzalkonium chloride, copper, silver, quaternary ammonium compounds, polyhexamethylene biguanide, chlorhexidine gluconate, fatty acid monoesters.

The attachment system also includes a polymeric hard good article for holding a vascular device, the polymeric hard good article having a first major surface and a second major surface. The first major surface is in contact with and attached to the second major surface of the base adhesive attachment layer, and the second major surface comprises a hole that passes through the first major surface, and a pathway adjacent to the hole. A variety of pathways are disclosed. In some embodiments, the pathway comprises a channel extending to an edge surface of the second major surface. In other embodiments, the pathway comprises a series of posts, wherein the series of posts define the pathway. Each of these embodiments is described in greater detail below.

A wide variety of polymeric materials are suitable for the hardgood articles of this disclosure. Typically, the hardgood article comprises a polymeric plate. In some embodiments, the hardgood article is optically transparent permitting easier monitoring of the vascular device. In other embodiments, the hardgood article is relatively opaque. The hardgood article is relatively rigid to provide support to the vascular device. Examples of suitable polymeric materials include polymethyl methacrylate (PMMA), and polycarbonate (PC).

The hardgood article can have a variety of sizes. In some embodiments, the hardgood has a thickness of from 1.5-3.5 mm. In some embodiments, the hardgood article is generally rectangular in shape with a length of 20-30 mm and a width of 20-30 mm.

The hardgood article includes a hole that passes through the entire hardgood article. The hole can have a wide range of shapes and sizes. The hole may be a symmetrical circular hole, or it may be a variety of other geometric shapes, such as for example hexagonal or octagonal, or it may be non-symmetrical such as oval shaped. Additionally, the hole may be connected to a chasm, that is to say a gap in the hardgood, that extends from the hole and may extend all the way to an edge of the article. The hole has a diameter that is suitable for holding vascular devices and is typically in the range of 1-4 millimeters.

The hardgood article also has a pathway on the second major surface of the hardgood article that is adjacent to the hole and extends to an edge of the hardgood article. The pathway constrains and holds the vascular device in place.

In some embodiments, the pathway comprises a channel, where the channel comprises a linear channel or a curved channel. The channel has a depth that is less than the thickness of the hardgood article, typically less than half the thickness of the hardgood article. It is desirable that the channel not be so deep that it weakens and impairs the rigidity of the hardgood article. The width of the channel is suitable for holding the tubing of vascular devices. Typically, the width of the channel is 1-4 millimeters.

The channel can have a variety of shapes. In some embodiments, the channel is a linear channel extending from an edge of the hardgood article to the hole. In other embodiments, the channel has a curved shape. A variety of curved shapes are suitable. In some embodiments, the curved channel comprises an S-shaped curved channel. In some embodiments, the pathway comprises a series of posts, wherein the series of posts define the pathway. As used herein, the term “posts” refers to elements that protrude from the surface of the hardgood article. These posts can have a wide variety of shapes such as straight posts comprising a shaft and a head, as well as a variety of shaped protrusions, where the protrusions can have the shape, for example, of a hook. The straight posts can have a variety of shapes, where the posts may be angled, of varying thickness, have a textured or smooth surface. Additionally, the heads can have a variety of shapes.

The pathway comprises at least two posts, typically more than two posts such that the pathway comprises a series of posts, where the series of posts form a tortuous pathway. The tortuous pathway helps to hold the tubing in place. In some embodiments, it is desirable that the posts comprise a material with a high coefficient of friction. Typical high coefficient of friction materials have a coefficient of friction of greater than 0.5. Like the tortuous path, the use of high coefficient of friction materials assists in holding the tubing in place.

The attachment system described above may further comprise a cover film article where the cover film article covers the attachment article described above to provide supportive attachment and to protect the vascular device from exposure to the environment. This can help to keep the vascular device site clean and free from contaminants.

The cover film article comprises a cover film with a first major surface and a second major surface, and a third adhesive layer, where the third adhesive layer has a first major surface and a second major surface. The second major surface of the third adhesive layer is in contact with the first major surface of the cover film, and the first major surface of the third adhesive layer is in contact with the second major surface of the polymeric hard good article.

Typically, it is desirable for the film of the film article to be optically transparent or even optically clear such that the underlying attachment article can be viewed and monitored. Suitable film materials include standard film-forming materials such as polyester, polyurethane, (meth)acrylate, polyolefin, or a combination thereof. Generally, the film has a thickness of greater than 20 micrometers (0.8 mil).

The third adhesive layer is typically a pressure sensitive adhesive. In some embodiments, it is desirable that the third adhesive layer be optically transparent or even optically clear such that the underlying attachment article can be viewed and monitored. A wide range of suitable adhesives are suitable. Pressure sensitive adhesives useful for the second adhesive layer include tackified natural rubbers, synthetic rubbers, tackified styrene block copolymers, polyvinyl ethers, acrylics, poly-alpha-olefins, and silicones. Example film articles include the TEGADERM line of products from 3M Company, St. Paul, MN.

As mentioned above, the attachment system of this disclosure is designed to attach a vascular device to a patient. A wide range of vascular devices are useful. Examples of suitable vascular devices include a PICC (Peripherally Inserted Central venous Catheter), a CVC (Central Venous Catheter) or other catheter.

As mentioned above, also disclosed herein are kits for securing vascular devices, where the kit comprises an attachment holding device and a cover film article. Attachment holding devices and cover film articles are described in detail above.

Also disclosed herein are methods for using an attachment system for vascular device. In some embodiments, the method comprises providing a vascular device, providing an attachment system for a vascular device,

Examples of vascular devices and attachment systems are described in detail above. In some embodiments, the attachment system comprises a base adhesive attachment layer with a first major surface and a second major surface, where the first major surface comprises an adhesive surface suitable for attachment to mammalian skin, and a polymeric hardgood article for holding a vascular device. The polymeric hardgood article has a first major surface and a second major surface, the first major surface being in contact with and attached to the second major surface of the base adhesive attachment layer. The second major surface comprises a hole that passes through the first major surface, and a pathway adjacent to the hole, the pathway comprising a channel extending to an edge surface of the second major surface, or a series of posts, where the series of posts define the pathway. In some embodiments, the hole may be connected to a chasm, that is to say a gap in the hardgood, that extends from the hole and may extend all the way to an edge of the article.

In some embodiments, typically those where the hardgood comprises a hole and does not comprise a chasm, the vascular device is threaded through the hole of the hardgood article, the vascular device is inserted into the patient, the securement device is adhered to the patient, and the vascular device is threaded through the pathway, whether a channel or series of posts.

In other embodiments, where the hardgood article comprises a hole and a chasm adjacent to the hole, the vascular device is inserted into the patient, the vascular device is passed through the chasm to the hole and is threaded through the hole of the hardgood article, the securement device is adhered to the patient, and the vascular device is threaded through the pathway, whether a channel or series of posts.

In some embodiments, the method further comprises providing a cover film article, where the cover film article comprises a cover film with a first major surface and a second major surface, and a third adhesive layer, where the third adhesive layer has a first major surface and a second major surface, where the second major surface of the third adhesive layer is in contact with the first major surface of the cover film. The first major surface of the third adhesive layer is contacted to the second major surface of the polymeric hard good article.

The current disclosure may be better understood in light of the figures showing embodiments of the current disclosure.

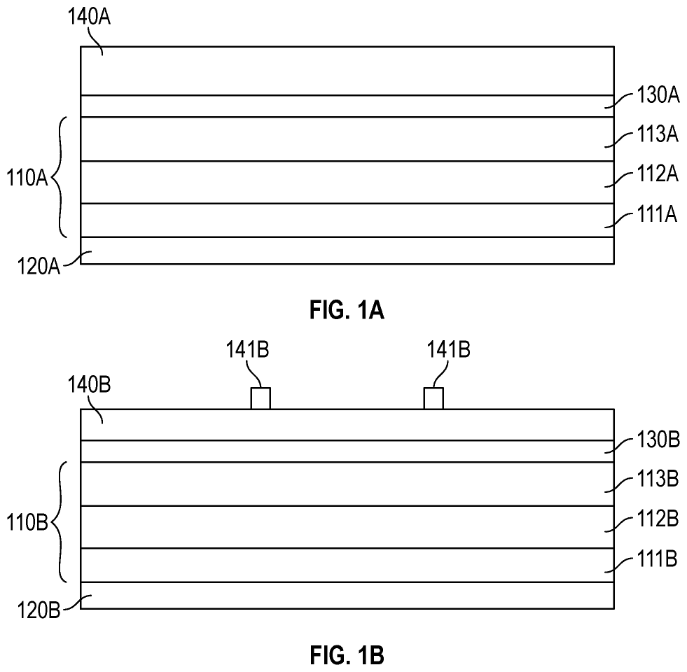

FIGS. 1A and 1B show cross-sectional views of articles of this disclosure. In the embodiments of FIG. 1A, the pathway comprises a channel (not shown). In the embodiments of FIG. 1B, the pathway comprises posts. FIG. 1A shows an embodiment of the base adhesive attachment layers and hardgood article of the vascular securement system of this disclosure. The base adhesive attachment layers comprise multi-layer extensible backing substrate 110A, first adhesive layer 120A, and second adhesive layer 130A. The multi-layer extensible backing substrate 110A comprises layers 111A, 112A, and 113A. Elastomeric core layer 112A is disposed between first skin layer 111A and second skin layer 113A. First adhesive layer 120A comprises a pressure sensitive adhesive layer suitable for attachment to mammalian skin, and second adhesive layer 130A attaches the multi-layer backing substrate 110A to hardgood article 140A. Hardgood article 140A contains a hole and channel that are not visible in this view.

FIG. 1B shows another embodiment of the base adhesive attachment layers and hardgood article of the vascular securement system of this disclosure. The base adhesive attachment layers comprise multi-layer extensible backing substrate 110B, first adhesive layer 120B, second adhesive layer 130B. The multi-layer extensible backing substrate 110A comprises layers 111B, 112B, and 113B. Elastomeric core layer 112B is disposed between first skin layer 111B and second skin layer 113B. First adhesive layer 120B comprises a pressure sensitive adhesive layer suitable for attachment to mammalian skin, and second adhesive layer 130B attaches the multi-layer backing substrate 110B to hardgood article 140B. Hardgood article 140B contains a hole not visible in this view and posts 141B.

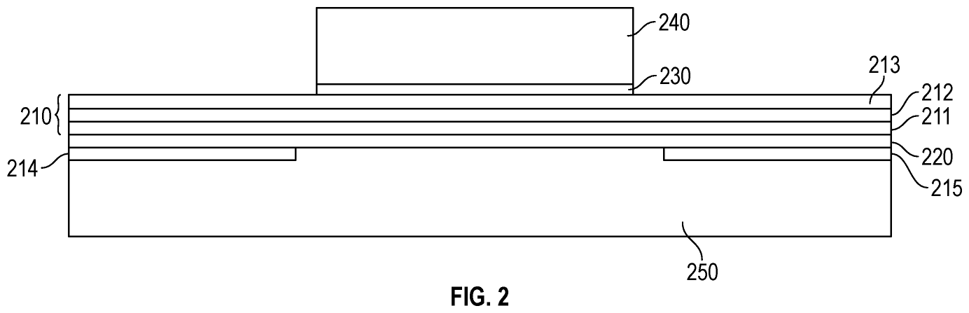

FIG. 2 shows an embodiment of an article attached to mammalian skin and containing tabs to facilitate the stretch removal of the base adhesive attachment layers. In FIG. 2, the attachment system comprises the base adhesive attachment layers and the hardgood article. The base adhesive attachment layers comprise multi-layer extensible backing substrate 210, first adhesive layer 220, and second adhesive layer 230. The multi-layer extensible backing substrate 210 comprises layers 211, 212, and 213. Elastomeric core layer 212 is disposed between first skin layer 211 and second skin layer 213. In the embodiment shown in FIG. 2, the non-tacky tabs 214 and 215 are disposed on first adhesive layer 220. The non-tacky tabs permit a user to grasp and stretch to remove the base adhesive attachment layers. First adhesive layer 220 comprises a pressure sensitive adhesive layer attached to the mammalian skin surface 250, and second adhesive layer 230 attaches the multi-layer backing substrate 210 to hardgood article 240. Hardgood article 240 contains a hole and channel that are not visible in this view. In other embodiments, not shown, the non-tacky tabs can be disposed on the first skin layer 21 or second skin layer 213.

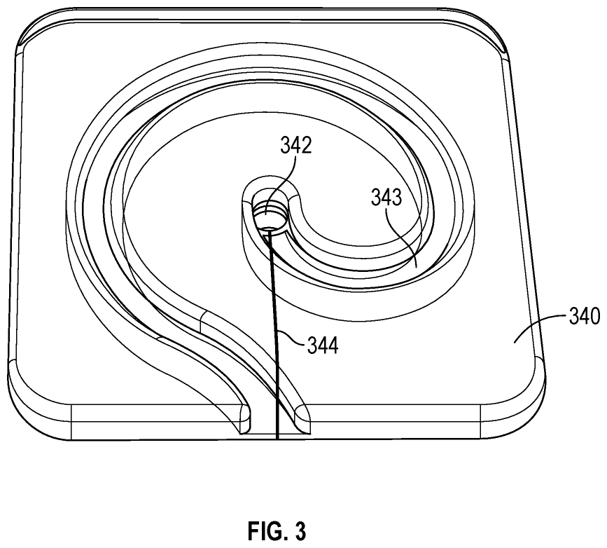

FIG. 3 is a top view of a hardgood article of this disclosure. Hardgood article 340 contains hole 342 and curved channel 343 and an optional slit 344 which connects the perimeter of article 340 to hole 342.

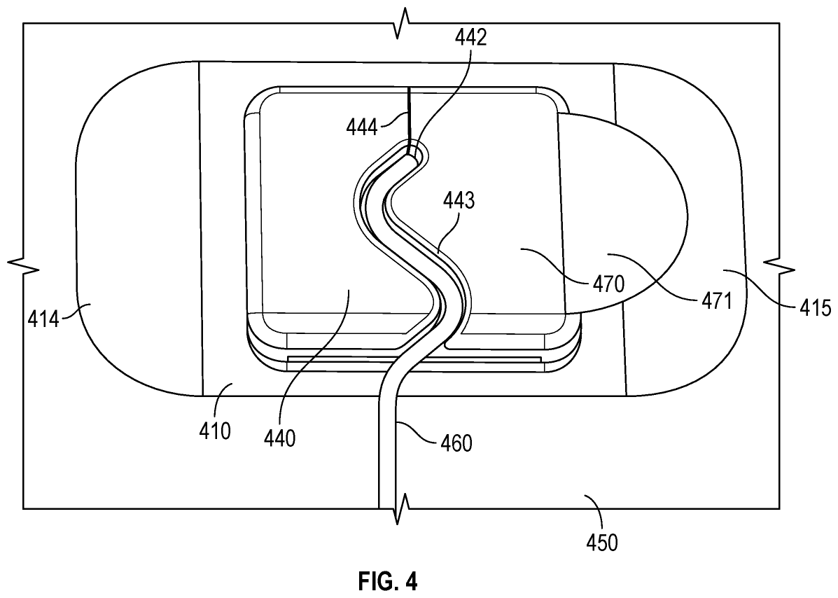

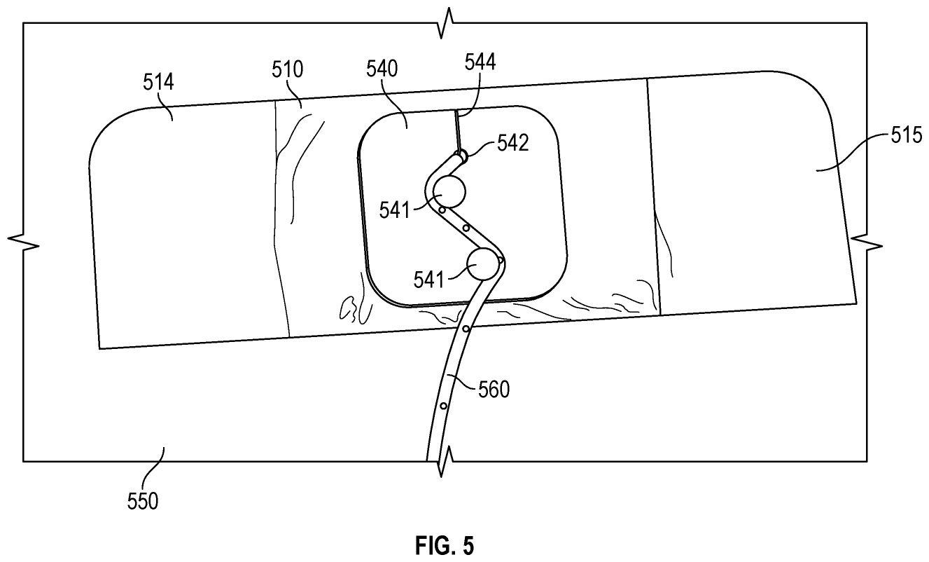

FIG. 4 is a top view of an article of this disclosure. The article is disposed on a test surface 450 and includes the multi-layer backing substrate with non-tacky tabs, hardgood article of the vascular securement system of this disclosure, cover film with non-tacky tab, as well as a vascular device. The multi-layer backing substrate 410 has non-tacky tabs 414 and 415 disposed upon it. Hardgood article 440 contains hole 442, curved channel 443, and slit 444. A portion of vascular device 460 is shown disposed in hardgood article 440. Cover film 470 with non-tacky tab 471 are adhered over the top of hardgood article 440 and vascular device 460. FIG. 5 is a top view of an article of this disclosure. The article is disposed on a test surface 550 and includes the multi-layer backing substrate with non-tacky tabs, hardgood article of the vascular securement system of this disclosure with posts, as well as a vascular device. The multi-layer backing substrate 510 has non-tacky tabs 514 and 515 disposed upon it. Hardgood article 540 contains posts 541, hole 542, and slit 544. A portion of vascular device 560 is shown disposed in hardgood article 440.

What is claimed is:

1. An attachment system for attaching a vascular device to a patient comprising:
a base adhesive attachment layer with a first major surface and a second major surface, the first major surface comprising an adhesive surface suitable for attachment to mammalian skin; and
a polymeric hard good article for holding a vascular device, the polymeric hard good article having a first major surface and a second major surface, the first major surface being in contact with and attached to the second major surface of the base adhesive attachment layer, and the second major surface comprises a hole that passes through the first major surface, and a pathway adjacent to the hole, the pathway comprising:
a channel extending to an edge surface of the second major surface; or
a series of posts, wherein the series of posts define the pathway.

2. The attachment system of claim 1, further comprising a cover film article, the cover film article comprising a cover film with a first major surface and a second major surface, and a third adhesive layer, wherein the third adhesive layer has a first major surface and a second major surface, wherein the second major surface of the third adhesive layer is in contact with the first major surface of the cover film, and the first major surface of the third adhesive layer is in contact with the second major surface of the polymeric hard good article.

3. The attachment system of claim 1, wherein the base adhesive attachment layer comprises a multi-layer stretch removable article comprising:
a multi-layer extensible backing substrate, wherein the multi-layer extensible backing substrate comprises at least three layers comprising:
a first plastic skin layer with a first major surface and a second major surface;
a core elastomeric layer with a first major surface and a second major surface;
and a second plastic skin layer with a first major surface and a second major surface, wherein the core elastomeric layer is in contact with the second major surface of the first plastic skin layer and the first major surface of the second plastic skin layer; and
a first adhesive layer in contact with the first plastic skin layer, wherein the first adhesive layer is suitable for attachment to mammalian skin; and
a second adhesive layer in contact with the third plastic skin layer, wherein the second adhesive layer attaches the multi-layer stretch removable article to the polymeric hard good article.

4. The attachment system of claim 3, wherein the core elastomeric layer comprises at least one of SEBS, SEPS, SIS, SBS, polyurethane, ethyl vinylacetate (EVA), ethyl methyl acrylate (EMA), ultra low linear density polyethylene (ULLDPE), hydrogenated polypropylene, and combinations or blends thereof.

5. The attachment system of claim 3, wherein the first or second skin layer comprises polypropylene, polyethylene, high density polyethylene (HDPE), low density polyethylene (LDPE), linear low density polyethylene (LLDPE), a polyurethane, EVA, EMA, and combinations or blends thereof.

6. The attachment system of claim 3, further comprising one or more tabs at a perimeter portion of the extensible substrate.

7. The attachment system of claim 1, wherein the pathway comprises a channel, wherein the channel comprises a linear channel, or a curved channel.

8. The attachment system of claim 7, wherein the curved channel comprises an S-shaped curved channel.

9. The attachment system of claim 1, wherein the pathway comprises a series of posts, wherein the series of posts form a tortuous pathway and comprise a material with a coefficient of friction of greater than 0.5.

10. The attachment system of claim 1, wherein the pathway comprises a series of posts, wherein the series of posts comprises 2 or greater posts.

11. The attachment system of claim 1, wherein at least one of the first or second adhesive layers comprises an anti-microbial adhesive layer.

12. The attachment system of claim 1, wherein the vascular device comprises a PICC (Peripherally Inserted Central venous Catheter), a CVC (Central Venous Catheter) or other catheter.

13. A kit for securing a vascular device comprising:
an attachable holding device comprising:
a base adhesive attachment layer with a first major surface and a second major surface, the first major surface comprising an adhesive surface suitable for attachment to mammalian skin; and
a polymeric hard good article for holding a vascular device, the polymeric hard good article having a first major surface and a second major surface, the first major surface being in contact with and attached to the second major surface of the base adhesive attachment layer, and the second major surface comprises a hole that passes through the first major surface, and a pathway adjacent to the hole, the pathway comprising:
a channel extending to an edge surface of the second major surface; or
a series of posts, wherein the series of posts define the pathway; and
a cover film article, the cover film article comprising:
a cover film with a first major surface and a second major surface, and a third adhesive layer, wherein the third adhesive layer has a first major surface and a second major surface, wherein the second major surface of the third adhesive layer is in contact with the first major surface of the cover film.

14. A method of using an attachment system for a vascular device comprising:
providing a vascular device;
providing an attachment system for a vascular device, the attachment system comprising:
a base adhesive attachment layer with a first major surface and a second major surface, the first major surface comprising an adhesive surface suitable for attachment to mammalian skin;
a polymeric hardgood article for holding a vascular device, the polymeric hard good article having a first major surface and a second major surface, the first major surface being in contact with and attached to the second major surface of the base adhesive attachment layer, and the second major surface comprises a hole that passes through the first major surface, and a pathway adjacent to the hole, the pathway comprising:
a channel extending to an edge surface of the second major surface; or
a series of posts, wherein the series of posts define the pathway;
inserting a vascular device into a patient, where the vascular device passes through the hole in the hardgood article;
attaching the attachment system to the patient; and
threading the vascular device through the pathway adjacent to the hole.

15. The method of claim 14, wherein inserting a vascular device into a patient comprises passing the vascular device through the hole of the hardgood article prior to inserting the vascular device into a patient.

16. The method of claim 14, wherein the hardgood article further comprises a chasm adjacent to the hole and inserting a vascular device into a patient comprises passing the vascular device through the chasm and into the hole of the hardgood article prior to inserting the vascular device into a patient.

17. The method of claim 14, further comprising:
providing a cover film article, wherein the cover film article comprises a cover film with a first major surface and a second major surface, and a third adhesive layer,
wherein the third adhesive layer has a first major surface and a second major surface,
wherein the second major surface of the third adhesive layer is in contact with the first major surface of the cover film; and
contacting the first major surface of the third adhesive layer to the second major surface of the polymeric hard good article.

18. The method of claim 14, wherein the pathway of the hardgood article comprises a channel and threading the vascular device through the pathway adjacent to the hole comprises inserting the vascular device into the channel.

19. The method of claim 14, wherein the pathway of the hardgood articles comprises at least two posts and threading the vascular device through the pathway adjacent to the hole comprises passing the vascular device around the posts.
